(19) United States
(12) Patent Application Publication
(10) Pub. No.: US 2015/0297615 A1
(43) Pub. Date: Oct. 22, 2015

(54) ANDROGEN RECEPTOR INACTIVATION CONTRIBUTES TO ANTITUMOR EFFICACY OF CYP17 INHIBITORS IN PROSTATE CANCER

(71) Applicant: UNIVERSITY OF MARYLAND, BALTIMORE, BALTIMORE, MD (US)

(72) Inventors: Vincent C.O. Njar, Glen Burnie, MD (US); Angela Brodie, Fulton, MD (US)

(21) Appl. No.: 14/791,155

(22) Filed: Jul. 2, 2015

Related U.S. Application Data
(63) Continuation of application No. 12/921,932, filed on Dec. 13, 2010.
(60) Provisional application No. 61/035,767, filed on Mar. 12, 2008.

(51) Int. Cl. A61K 31/58

(52) U.S. Cl. CPC A61K 31/58

(57) ABSTRACT

Provided are methods of inhibiting CYP17 in a mammal, such as a human, that include administering an effective amount of at least one CYP17 inhibitor, such as VN/124-1, VN/125-1, VN/85-1, VN/87-1 and/or VN/108-1 to the mammal. Also provided are methods of down regulating androgen receptor (AR) protein expression and methods of antagonizing AR in a mammal that include administering to the mammal an effective amount of at least one active ingredient selected from VN/124-1, VN/125-1, VN/85-1, VN/87-1 and VN/108-1. Also provided are methods of treating prostate cancer and methods of suppressing or preventing prostate tumor growth by administering such compounds to a mammal.

### STATEMENT OF RELATED APPLICATION

This patent application claims the benefit of priority to U.S. Provisional Patent Application No. 61/035,767 filed on Mar. 12, 2008, entitled “Androgen Receptor Inactivation Contributes to Antitumor Efficacy of CYP17 Inhibitor VN/124-1 in Prostate Cancer,” which is hereby incorporated by reference in its entirety, including all text and figures.

### STATEMENT REGARDING FEDERALLY SPONSORED RESEARCH

This invention was made with government support under NIH Grant No. CA27440 awarded by the National Institutes of Health. The U.S. government has certain rights in the invention.

### TECHNICAL FIELD

The present embodiments relate generally to methods for treatment and/or prevention of prostate cancer in mammals. By way of non-limiting example, the present embodiments include methods of treating and preventing prostate tumor growth in humans.

### BACKGROUND ART

Prostate cancer (PC) is the most prevalent cancer in men and the second leading cause of death in American men resulting in 218,890 new cases and 27,050 deaths per year from this disease [1]. Androgens play an important role in the development, growth, and progression of PC [2]. The testes produce most of the circulating testosterone (T), whereas approximately 10% is synthesized by the adrenal glands. T is further converted in the prostate to the more potent androgen dihydrotestosterone (DHT) by the enzyme 5α-reductase [3]. Most PCs are initially dependent on androgens for their growth, and orchidectomy (either surgical or medical with a GnRH agonist) remains the standard of treatment. Although orchidectomy reduces androgen production by the testes, androgen synthesis in the adrenal glands is unaffected. Thus, orchidectomy combined with antiandrogens to block the action of adrenal androgens can be more effective and prolongs survival of PC patients [4].

The mechanisms through which androgen-dependent PC tumors survive and proliferate under androgen deprivation therapy (ADT) are not completely understood. However, it has been found that the androgen receptor (AR) is consistently expressed and active in multiple xenograft models of hormone refractory disease[5]. Amplified expression and increased sensitivity of AR in recurrent PC may be due to its increased stability, altered growth factor signaling, and mutations that broaden ligand specificity [6-9]. Additionally, reduction of AR expression in androgen sensitive and refractory models through the use of shRNA, or chemical means, have resulted in marked growth suppression of PC cells [10-13]. Further support for the role of AR and androgens in PC is the recent report of increased expression of genes of androgen converting enzymes and persistence of androgen regulated genes in androgen-independent PC [14-16]. These observations suggest that therapies that inhibit production of androgens and target multiple points in the AR signaling cascade could offer a more effective approach for prolonging remission of PC.

In the testes and adrenal glands, the last step in the biosynthesis of T involves two key sequential reactions, that are catalyzed by a single enzyme, the cytochrome P450 monooxygenase 17α-hydroxylase/17,20-lyase (CYP17) [17]. Ketoconazole, an antifungal agent and non-specific CYP450 inhibitor used with careful scheduling [18] can produce prolonged responses in otherwise hormone-refractory PC patients. Furthermore, ketoconazole was found to retain activity in advanced PC patients with progression despite flutamide withdrawal [19]. Although ketoconazole remains one of the most effective second line hormonal therapies for PC, its use is limited due to liver toxicity and other side effects. However, its antitumor efficacy suggests that more potent and selective inhibitors of CYP17 could provide useful agents for treating this disease [20].

### SUMMARY

The present inventors, and others, have reported a number of novel inhibitors of CYP17, and some have been shown to be strong inhibitors of testosterone production in rodent models [20-23]. Jarman and colleagues recently described the effects of their steroidal CYP17 inhibitor, abiraterone, in patients with PC [24, 25]. Some of the most effective CYP17 inhibitors possess additional activities, such as inhibition of 5α-reductase and/or are antiandrogens with potent antitumor efficacy [26-29].

The present inventors have demonstrated that CYP17 inhibitors, including VN/124-1, possess several anti-cancer properties that target androgen receptor (AR).

In addition to being among the strongest CYP17 inhibitors known to date, the novel steroidal compounds VN/85-1, VN/87-1 and VN/108-1 were shown to reduce DHT stimulated LNCaP cell proliferation, and displace methyltrienolone (R1881), a synthetic androgen, from the mutated T877A AR at a 5 μM concentration [26]. VN/124-1 (FIG. 1) was found to be effective in vitro as well as in the LAPC4 xenograft model in male SCID mice [28]. In addition to inhibition of CYP17, VN/124-1 exhibited potent AR antagonism in binding studies and LNCaP luciferase transcription assays, as well as marked tumor growth suppression in LAPC4 xenografts [28].

In the present application, the present inventors demonstrate that VN/124-1 and other novel CYP17 inhibitors cause down-regulation of AR protein expression in vitro and in vivo. This mechanism of action appears to contribute to their antitumor efficacy. It was also shown by comparison that the in vivo antitumor efficacy of VN/124-1 with that of castration demonstrated that VN/124-1 is more potent than castration in human PC xenograft models.

In example embodiments, methods are drawn to inhibition of CYP17 by administering at least one of VN/124-1, VN/125-1, VN/85-1, VN/87-1 and VN/108-1. In other embodiments, methods are drawn to antagonizing AR by administering at least one of such compound(s). In further other embodiments, example methods are drawn to down regulating AR protein by administering at least one of such compound(s). In specific embodiments, AR downregulation occurs by particular cellular processes, including those that result in AR degradation. In other embodiments, methods are drawn to a combination of inhibition of CYP17, AR antagonism, and AR downregulation.

In example embodiments, methods are provided for treating prostate cancer by administering at least one CYP17 inhibitor to a mammal having such prostate cancer, where the CYP17 inhibitor includes at least one of VN/124-1, VN/125-1, VN/85-1, VN/87-1 and VN/108-1. Example embodiments are drawn to methods of suppressing or preventing prostate tumor growth in a mammal by administering to the mammal at least one of VN/124-1, VN/125-1, VN/85-1, VN/87-1 and VN/108-1.

The foregoing has outlined rather broadly the features and technical advantages of the present invention in order that the detailed description of the invention that follows may be better understood. Additional features and advantages of the invention will be described herein, which form the subject of the claims of the invention. It should be appreciated by those skilled in the art that any conception and specific embodiment disclosed herein may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that any description, figure, example, etc. is provided for the purpose of illustration and description only and is by no means intended to define the limits the invention.

### BRIEF DESCRIPTION OF THE DRAWINGS

Non-limiting example embodiments described herein, with reference to the following accompanying Figures.

FIG. 1 depicts the chemical structure of 3β-hydroxy-17-(1H-benzimidazole-1-yl)androsta-5,16-diene (VN/124-1).

FIG. 2 depicts the effect of compounds on DHT stimulated transcription. LAPC4 cells were transfected with the ARR-2 reporter construct+the Renilla luciferase reporting vector pRL-null and treated with novel compounds for 18 hours in the presence of 10 nM DHT. Control represents baseline activity without androgen stimulation. Androgen stimulated luciferase activity (luminescence) was measured in a Victor 1420 plate reader. The results are presented as the fold induction, that is, the relative luciferase activity of the treated cells divided by that of the control, normalized to that of the Renilla.

FIG. 3A depicts a Western Blot Analysis of AR expression in vitro. Cells were treated with test compounds for 24 hours at the indicated concentrations (1-15 μM). Cell extracts were prepared and probed with anti AR and anti β-actin antibodies. AR expression in LNCaP cells after 24 hr treatment with the indicated compounds. (FIG. 3B). Densitometry quantification of AR expression in LAPC4 cells after treatment with 15 μM of the indicated compounds. (FIG. 3C). Densitometry quantification of AR expression in LNCaP cells after treatment. (FIG. 3D)

FIG. 4 depicts effects of VN/124-1, casodex, and castration on the prevention and growth of LAPC4 human prostate xenografts in male SCID mice. Mice bearing LAPC4 human prostate tumors were grouped and treatment started 63 days after cell inoculation except for the “prevention” group. In this group, treatment was begun with VN-124-1 on the day of cell inoculation. Treatments with both casodex and VN/124-1 were given at a dosage of 0.13 mmol/kg/twice daily. Control mice (vehicle treated mice were sacrificed after 86 days because of large tumors and mice treated with casodex were sacrificed due to insufficient drug. Tumors of all treated groups were significantly different from control and the “prevention” group was also significantly different from all treated groups. *VN/124-1 alone and VN/124-1 plus castration were significantly different from castration and from casodex using multivariative analysis.

FIG. 5 depicts percent change in mouse body weight over treatment duration in LAPC4 human prostate cancer xenografts in male SCID mice.

FIG. 6 is a Western immunobloting analysis of whole cell lysates from LAPC4 tumors following various treatments.

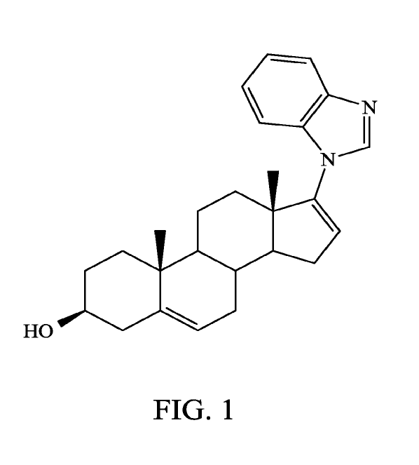

### DETAILED DESCRIPTION

Generally provided herein are various methods that may be useful for treatment and/or prevention of prostate cancer in mammals. By way of non-limiting example, the present embodiments may include methods of treating or preventing prostate cancer in humans.

Additional aspects, advantages and/or other features of example embodiments will become apparent in view of the following detailed description, taken in conjunction with the accompanying drawings. It should be apparent to those skilled in the art that the described embodiments provided herein are merely exemplary and illustrative and not limiting. Numerous embodiments of modifications thereof are contemplated as falling within the scope of this disclosure and equivalents thereto.

All patents and publications mentioned in this specification are indicative of the level of those skilled in the art to which the invention pertains. All patents and publications herein are incorporated by reference to the same extent as if each individual publication was specifically and individually indicated as having been incorporated by reference in its entirety.

Unless otherwise noted, technical terms are used according to conventional usage. Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar technical references.

As used herein, “a” or “an” may mean one or more. As used herein in the claim(s), when used in conjunction with the word “comprising”, the words “a” or “an” may mean one or more than one. As used herein “another” may mean at least a second or more. Furthermore, unless otherwise required by context, singular terms include pluralities and plural terms include the singular.

The novel compound 3β-hydroxy-17-(1H-benzimidazole-1-yl)androsta-5,16-diene (VN/124-1) is a potent CYP17 inhibitor/antiandrogen and strongly inhibits the formation and proliferation of human prostate cancer LAPC4 tumor xenografts in SCID mice. In certain aspects VN/124-1 and other novel CYP17 inhibitors also cause down-regulation of AR protein expression. This mechanism of action appears to contribute to their antitumor efficacy. The present inventors compared the in vivo antitumor efficacy of VN/124-1 with that of castration and a clinically used antiandrogen, casodex, and showed that VN/124-1 is more potent than castration in LAPC4 xenograft model. Treatment with VN/124-1 (0.13 mmol/kg/twice daily) was also very effective in preventing the formation of LAPC4 tumors (6.94 vs. 2410.28 mm³ in control group). VN/124-1 (0.13 mmol/kg/twice daily) and VN/124-1 (0.13 mmol/kg/twice daily)+castration induced regression of LAPC4 tumor xenografts by 26.55% and 60.67%, respectively. Treatments with casodex (0.13 mmol/kg/twice daily) or castration caused significant tumor suppression compared with control. Furthermore, treatment with VN/124-1 caused marked down-regulation of AR protein expression, in contrast to treatments with casodex or castration that caused significant up-regulation of AR protein expression. In certain aspects VN/124-1 acts by several mechanisms (CPY17 inhibition, competitive inhibition, and down-regulation of the androgen receptor). These actions contribute to inhibition of the formation of LAPC4 tumors and cause regression of growth of established tumors. VN/124-1 is more efficacious than castration in the LAPC4 xenograft model demonstrating the compound has potential for the treatment of prostate cancer.

Example methods provided herein include methods of inhibiting CYP17 in a mammal, such as a human, by administering an effective amount of at least one CYP17 inhibitor to the mammal. Non-limiting example CYP17 inhibitors may include at least one of VN/124-1, VN/125-1, VN/85-1, VN/87-1 and VN/108-1. As discussed herein and shown in the accompanying figures, example methods include administration of VN/124-1 as the CYP17 inhibitor. Other example methods include administration of at two or more CYP17 inhibitors.

VN/124-1, VN/125-1, VN/85-1, VN/87-1 and VN/108-1, including the synthesis of such compounds, are discussed e.g., in WO2006093993 filed Mar. 2, 2006, which claims priority to U.S. Provisional application 60/657,390 filed Mar. 2, 2005, the contents of both of which are incorporated herein by reference in their entirety.

Active ingredients provided herein may be administered to a mammal, as part of a composition. Example compositions may optionally include one or more excipients or active ingredients as would be apparent to those skilled in the art. The term “excipient” is used herein to include pharmaceutically acceptable inert substances added to a drug formulation to give e.g., a desired consistency or form.

The term “active ingredients” is used herein to include any drug or other active ingredient that may be used for treating mammals for a variety of different conditions including prostate cancer. The term is not meant to be limiting, and may include any “active ingredient” and “drug” known to those skilled in the art, which may be administered in the present methods.

The terms “active ingredients” and “drugs” are also intended to encompass analogs, prodrugs, salts, esters, polymorphs, and/or crystalline forms of active ingredients and drugs, as would be apparent to those skilled in the art.

The active ingredient, such as one or more CYP17 inhibitor(s) or compositions including any active ingredients may be administered by methods known to those skilled in the art including, but not limited to, intraperitoneally, intravenously, orally, subcutaneously, intradermally, intramuscularly, intravascularly, endotracheally, intraosseously, intra-arterially, intravesicularly, intrapleurally, topically, intraventricularly, or through a lumbar puncture (intrathecally).

Those skilled in the art would be able to determine the appropriate effective amount of active ingredient(s), such as VN/124-1, for administration in the various methods herein, depending on various factors, including but not limited to, the desired result to be achieved by the method, the type of mammal to whom the ingredient is being administered (e.g., human), the weight of the mammal, and the composition, formulation and/or method of administration.

Example methods provided herein also include methods of down regulating AR protein expression in a mammal, such as a human, by administering an effective amount of at least one active ingredient selected from the group consisting of VN/124-1, VN/125-1, VN/85-1, VN/87-1 and VN/108-1. According to non-limiting example embodiments, the active ingredient includes VN/124-1. Other example methods include administration of at two or more active ingredients. Example methods may include for example, methods of down regulating androgen receptor protein expression in LNCaP cells or LAPC4 cells.

Example methods further include methods of antagonizing AR in a mammal, such as a human, by administering an effective amount of at least one active ingredient selected from the group consisting of VN/124-1, VN/125-1, VN/85-1, VN/87-1 and VN/108-1, to the mammal. According to non-limiting example embodiments, the active ingredient includes VN/124-1. Other example methods include administration of at two or more active ingredients.

Further examples may include at least one of inhibiting CYP17, downregulating AR protein and/or antagonizing AR expression in a mammal by administering at least one CYP17 inhibitor, such as VN/124-1, VN/125-1, VN/85-1, VN/87-1 and VN/108-1, to the mammal. According to non-limiting example embodiments, the active ingredient comprises VN/124-1.

Also provided are methods of suppressing or inhibiting tumor growth in a mammal, such as a human, having at least one tumor, which methods include administering an effective amount of at least one active ingredient selected from the group consisting of VN/124-1, VN/125-1, VN/85-1, VN/87-1 and VN/108-1, to the mammal. According to non-limiting example embodiments, the active ingredient comprises VN/124-1. Other example methods include administration of at two or more active ingredients. According to non-limiting example embodiments, tumors may include a LAPC4 human prostate tumor.

Provided herein are methods of treating a mammal, such as a human, having prostate cancer, which methods include administering an effective amount of at least one active ingredient selected from the group consisting of VN/124-1, VN/125-1, VN/85-1, VN/87-1 and VN/108-1, to the mammal. According to non-limiting example embodiments, the active ingredient comprises VN/124-1. Other example methods include administration of at two or more active ingredients.

Further provided herein are methods of preventing human prostate tumors from forming in a mammal, such as a human, which methods include administering an effective amount of at least one active ingredient selected from the group consisting of VN/124-1, VN/125-1, VN/85-1, VN/87-1 and VN/108-1, to the mammal. According to non-limiting example embodiments, the active ingredient comprises VN/124-1. Other example methods include administration of at two or more active ingredients.

Applicants note that the above methods are not necessarily independent from one another. By way of non-limiting example, although methods of inhibiting tumor growth and methods of down-regulating AR protein expression are each described herein, they are not necessarily separate from one another, rather tumor growth may be inhibited as a result of the down-regulation of AR protein expression.

Non-limiting example embodiments are directed to methods that include administering one of the following active ingredients: VN/124-1, VN/125-1, VN/85-1, VN/87-1 or VN/108-1. Other, non-limiting example embodiments are directed to methods that include administering more than one active ingredient selected from VN/124-1, VN/125-1, VN/85-1, VN/87-1 and VN/108-1.

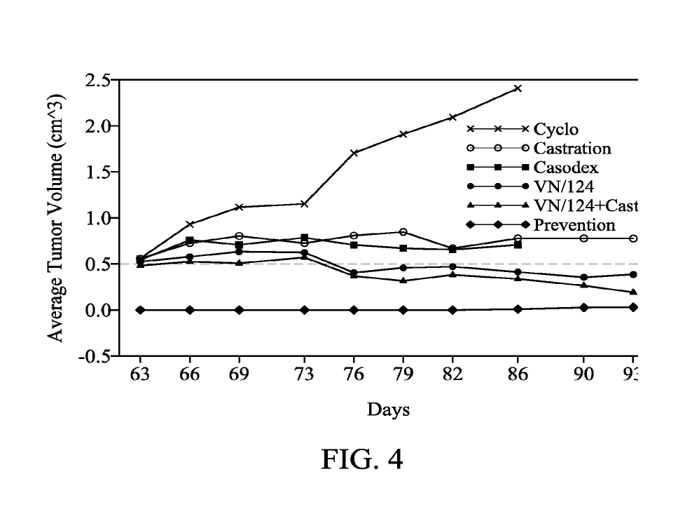

The methods provided herein are not intended to be limited to administration of the one or more recited compounds or compositions containing such compound(s), but may further include the administration of additional compounds, compositions and/or treatments to try to achieve the goal of each method, as would be apparent in view of the present disclosure and the knowledge of those skilled in the art. By way of non-limiting example, certain example methods herein include suppressing tumor growth by administering to a mammal an effective amount e.g., VN/124-1. Such methods may further include for example, castrating the mammal or other techniques that may further achieve the desired goal of suppressing tumor growth (See e.g., FIG. 4).

Example embodiments are also directed to methods that include administering at least one anti-tumor or anti-cancer agent, in addition to the one or more active ingredients selected from VN/124-1, VN/125-1, VN/85-1, VN/87-1 and VN/108-1. The additional anti-tumor or anti-cancer may be selected from agents known to these skilled in the art. Non-limiting example embodiments may include for example agents that may be effective at preventing and/or treating prostate tumors or prostate cancer.

Materials:

Casodex (Bicalutamide) was provided by Dr. E. Anderson from Astra Zeneca UK. The compounds VN/124-1, VN/125-1, VN/85-1, VN/87-1 and VN/108-1 were synthesized in our laboratory as described previously [26, 28]. AR antibody (SC-7305) was obtained from Santa Cruz Biotechnology (Santa Cruz, Calif.). Tritiated methyltrienolone ([³H]R1881) was obtained from Perkin Elmer LAS (Waltham, Mass.).

Cell Culture:

LAPC4 cells were grown in IMEM supplemented with 15% FBS, 1% penicillin/streptomycin solution, and 10 nM DHT. LNCaP cells were maintained in RPMI 1640 medium supplemented with 10% FBS and 1% penicillin/streptomycin solution. PC-3-AR cells were grown in the same media supplemented with 750 μg/ml G418, for continued selection of the AR vector.

DNA Constructs and Transfections:

The Probasin luciferase reporter construct ARR2-Luc was generated by insertion of the minimal probasin promoter ARR2, into the polyclonal linker region of PGL3-enhancer vector (Promega) as described previously [30]. The pRL-null (Promega) was used as the internal control. PC-3 cells stably transfected with the human wild-type AR (designated PC-3AR), and the T575A human AR mutation vector were kindly provided by Dr. Marco Marcelli (Baylor College of Medicine, Houston, Tex.; [31]. All transfections were carried out utilizing LipofectAMINE 2000 transfection reagent (Invitrogen) according to the manufacturer's protocol.

Competitive Binding Assays:

In order to determine if the CYP17 inhibitors interact with the AR, competitive binding assays were performed as described previously [28]. The ability of the test compounds (1 nM-10 μM) to displace [³H]R1881 (5.0 nM) from the AR was determined in LAPC4 cells (wild-type AR), PC3 cells transfected with wild-type AR (PC3-AR), LNCaP cells which express an endogenous AR with a mutation in the ligand binding domain (T877A), and PC3 cells transfected with an AR containing a mutation in the DNA binding domain (T575A).

Transcriptional Activation—Luciferase Assay:

LAPC4 and LNCaP cells were transferred to steroid-free medium 3 days before the start of the experiment, and plated at 1×10⁵ cells/well in steroid-free medium. The cells were dual transfected with ARR2-Luc and the Renilla luciferase reporter vector pRL-null as described in DNA Constructs and Transfections. After a 24 hour incubation period at 37° C., the cells were incubated in fresh phenol-red free RPMI 1640 medium containing 5% charcoal-stripped FBS and treated with 10 nM DHT, ethanol vehicle and/or the selected compounds in triplicate. After an 18 hour treatment period, the cells were washed twice with ice-cold DPBS and assayed using the Dual Luciferase kit (Promega) according to the manufacturer's protocol. Cells were lysed with 100 μl of luciferase lysing buffer, collected in a microcentrifuge tube, and pelleted by centrifugation. Supernatants (20 μl aliquots) were transferred to corresponding wells of opaque 96-well multiwell plates. Luciferase Assay Reagent was added to each well, and the light produced during the luciferase reaction was measured in a Victor 1420 scanning multi-well spectrophotometer (Wallac, Inc., Gaithersburg, Md.). After measurement, Stop and Glo reagent (Promega) was added to quench the firefly luciferase signal and initiate the Renilla luciferase luminescence. Renilla luciferase luminescence was also measured in the Victor 1420. The results are presented as the fold induction, that is, the relative luciferase activity of the treated cells divided by that of the control, normalized to that of the Renilla.

AR Down-Regulation and Degradation:

In order to determine the ability of the test compounds to modulate AR protein levels, LAPC4 and LNCaP cells were treated with concentrations ranging from 1-15 μM for 24 hours. Cells were collected and lysates prepared. Equal amounts of total protein were analyzed for AR expression levels by western blot analysis. Equal amounts of total protein (50-100 μg) were subjected to sodium dodecyl sulfate-polyacrylamide gel electrophoresis (SDS-PAGE 60 V, 3 hr) and transferred (90 V, 1 hr) to nitrocellulose membranes (Hybond ECL, Amersham). Immunodetections were performed using mouse monoclonal antibody against human AR (SC-7305 Santa Cruz Biotechnologies, Inc, Santa Cruz, Calif.). Immunoreactive bands were visualized using the enhanced chemiluminescence detection reagents (Amersham Corp., Arlington Heights, Ill.) according to the manufacturer's instructions and quantitated by densitometry using ImageQuant 5.0 software.

For degradation studies, LNCaP cells were grown in serum free medium for three days, and treated with 10 μM cycloheximide alone, 15 μM VN/124-1 alone, or 10 μM cycloheximide+15 μM VN/124-1 for 0, 2, 6, 12, and 24 hours. Cells were collected by centrifugation and the cell pellet was resuspended in chilled lysis buffer [0.1M Tris HCl, 0.5% TritonX-100, protease inhibitors (Complete™, Boehringer, Indianapolis, Ind.)] and sonicated for 20 seconds. The homogenates were transferred to Eppendorf tubes, incubated on ice for 30 minutes, and then spun at 14,000 rpm for 20 minutes. The supernatants were stored at −70° C. Western bolts were performed as above. Protein concentrations were determined with a Bio-Rad kit (BioRad, Hercules and CA).

Cell Proliferation Assay:

To determine the effect of steroids and novel compounds on cell proliferation, each cell type was transferred into steroid-free medium three days prior to the start of the experiments (steroid-free medium consisted of phenol red free RPMI supplemented with 5% dextran-coated, charcoal treated serum and 1% penicillin/streptomycin solution). Growth studies were then performed by plating cells (1.5×10⁴cells/well) in 24-well multiwell dishes (Corning, Inc. Corning, N.Y.). After a 24 hour attachment period, the medium was aspirated and replaced with steroid-free medium containing vehicle or the indicated concentrations of androgens and novel compounds (1 nM-10 μM). Control wells were treated with vehicle (ethanol). Casodex (bicalutamide) was used as a reference drug for comparison to a known anti-androgen. The medium was changed every three days and the numbers of viable cells were compared by MTT or XTT (LNCaP) assay on the seventh day.

For the MTT procedure, following incubation of cells for the above mentioned time, 0.5 mg/ml MTT was added to each well and incubated at 37° C. for three hours. Following incubation, the medium was aspirated completely, with care taken not to disturb the formazan crystals. DMSO (500 μl) was used to solubilize these crystals. After slight shaking, the plates were read at 540 nM with a Victor 1420 scanning multi-well spectrophotometer. All results represent the average of a minimum of three wells. Additional control consisted of medium alone with no cells. XTT was performed essentially as MTT, with the deletion of the solubilization step and was preferred for the LNCaP cells that adhere poorly to the plates. A water soluble formazan was obtained using XTT, and the plates were read at 450 nM with the spectrophotometer.

In Vivo Antitumor Studies (LAPC4 Prostate Cancer Xenografts):

All animal studies were performed according to the guidelines and approval of the Animal Care Committee of the University Of Maryland School Of Medicine, Baltimore. Male severe combined immunodeficient (SCID) mice 4-6 weeks of age purchased from the National Cancer Institute, (Fredrick, Md.) were housed in a pathogen-free environment under controlled conditions of light and humidity and allowed free access to food and water. Tumors were developed from LAPC4 cells inoculated subcutaneously (s.c.) into each mouse as previously described [28]. LAPC4 cells were grown in IMEM with 15% FBS plus 1% PS and 10 nm DHT until 80% confluent. Cells were scraped into DPBS, collected by centrifugation and resuspended in Matrigel (10 mg/mL) at 3×10⁷ cells/mL. Mice were injected s.c. with 100 μL of the cell suspension at one site on each flank. Twice per week, the mice were weighed and tumors were measured with calipers for the duration of experiment. Tumor volumes were calculated by the formula: 4/3π×r₁²×r₂ (r₁<r₂). Mice in the tumor formation prevention group (n=5) were injected with VN/124-1 (0.13 mmol/kg/twice daily) in the vehicle (0.3% saline hydroxypropyl cellulose; HPC) from the day of inoculation for the duration of the experiments. The rest of the mice were monitored until tumors reached approximately 500 mm³, about 9 weeks after cell inoculation. Mice were assigned to five groups (five mice per group) for treatment so that there was no statistically significant difference in tumor volumes amongst the groups at the beginning of the treatment. The five groups were: control, castration, casodex (0.13 mmol/kg/twice daily), VN/124-1 (0.13 mmol/kg/twice daily), and VN/124-1 (0.13 mmol/kg/twice daily)+castration. Compounds (suspensions in 0.3% PHC) were given s.c. twice daily, 9 a.m. and 5 p.m. Mice were castrated under methoxyfluorane anesthesia. Control and castrated mice were treated with vehicle (HPC) only. At the end of the treatment period, the animals were sacrificed under flurothane anesthesia; tumors were excised, weighed and stored at −80° C. The liver and kidneys were also harvested and examined for any abnormalities. Animals were also monitored for general health status and signs of possible toxicity due to treatment.

Tumor Analysis:

The protein extracts of the tumors from the above experiment were prepared by homogenizing the tissue in ice-cold DPBS containing protease inhibitors. Western blots were performed as described above.

Statistical Analysis:

The total tumor volume of each group was compared using the log scale. Because of incomplete values, the quasi-t-test developed by Tan et al. [32] for comparison between two groups was used. Total tumor volume was compared from day 63 to day 93, the total tumor volume from day 76 to day 93 (the treatment effect after two weeks), and at day 93. All computations were performed using S-PLUS. The treatment groups were compared with one another at 0.05 level of significance.

The following examples are provided to further illustrate various non-limiting embodiments and techniques. It should be understood, however, that these examples are meant to be illustrative and do not limit the scope of the claims. As would be apparent to skilled artisans, many variations and modifications are intended to be encompassed within the spirit and scope of the invention.

### Experimental Examples

### Competitive Binding to Wild Type and Mutant Androgen Receptors

LNCaP cells expressed a single class of high-affinity binding sites with K_{d}=0.5 nM, and Bₘₐₓ determined as 1.18×10⁵ sites/cell. LAPC4 cells had a similar K_{d} of 0.4 nM with a Bₘₐₓ of 6.1×10⁴ sites/cell. Once the saturation concentration (5 nM) was determined, evaluation of the compounds previously tested at 5 μM in LNCaP cells, VN/85-1, VN/87-1, VN/108-1 [33], was conducted over a full concentration range in both cell types. Casodex, an anti-androgen currently used as PC therapy, was included as a reference drug (See Table 1 below). Abiraterone, a CYP17 inhibitor currently in clinical trials, was also tested.

TABLE 1
Competitive inhibition of [³H]R1881 binding
(LAPC4, PC3-AR, PC3-ART575A, and LNCaP cells)

| | wild-type (IC₅₀ nM) | T877A (IC_{50 nM}) | T575A (IC₅₀ nM) |
|---|---|---|---|
| Compound | (LAPC4/PC3-AR) | (LNCaP) | (PC3-ART575A) |
| VN/85-1 | 341 | 1290 | 473 |
| VN/87-1 | 319 | 422 | NT |
| VN/108-1 | 868 | 831 | 1210 |
| VN/124-1 | 405 | 845 | 454 |
| VN/125-1 | 248 | 1240 | 383 |
| Abiraterone | — | — | NT |
| Casodex | 4300 | 971 | NT |
| Flutamide | 10985 | 11600 | NT |

NT = Not tested,
— = less than 20% inhibition at 10 μM

In particular, Table 1 depicts the affinity of various compounds for the AR. The ability of the test compounds to displace [³H]R1881 (5.0 nM) from the androgen receptor was determined in LAPC4 cells (wild-type AR), PC3 cells transfected with wild-type AR, LNCaP cells which express an endogenous AR with a mutation in the ligand binding domain (T877A), and PC3 cells transfected with an AR containing a mutation in the DNA binding domain (T575A). Cells were plated (2-3×10⁵) in 24 well multiwell dishes in steroid-free medium and allowed to attach. The following day the medium was replaced with serum-free, steroid free RPMI supplemented with 0.1% BSA and containing [³H]R1881 (5 nM), test compounds (1 nM-10 μM), and 1 μM triamcinolone acetonide to saturate progesterone and glucocorticoid receptors. Following a 2 hour incubation period at 37° C., cells were washed twice with ice-cold DPBS and solubilized in DPBS containing 0.5% SDS and 20% glycerol. Extracts were removed and cell associated radioactivity counted in a scintillation counter. The data was analyzed by nonlinear regression using Graphpad Prism software (GraphPad Software, Inc, San Diego, Calif.).

VN/85-1, VN/87-1, VN/124-1, and VN/125-1 had the highest affinity for the wild-type AR. In contrast abiraterone did not bind to the AR. There was no significant difference found between the wild-type AR in transfected PC3-AR cells and the wild-type AR expressed endogenously in LAPC4 cells (Table 1). VN/85-1, VN/124-1, and VN/125-1 had slightly greater ability to displace [³H]R1881 from the wild-type receptor than from the T877A mutant in LNCaP cells. The T877A AR contains a mutation in the ligand binding domain [34] which could account for the observed difference in binding. In contrast, VN/108-1 and VN/87-1 demonstrated nearly identical affinities for both receptor types. PC3-T575-AR cells express an AR with a mutation in the DNA binding domain [31]. Unlike the T877A AR, there was no apparent difference observed in VN/85-1, VN/124-1, or VN/125-1's ability to displace [³H]R1881 from the T575A AR when compared with wild-type.

Androgen Receptor Antagonism:

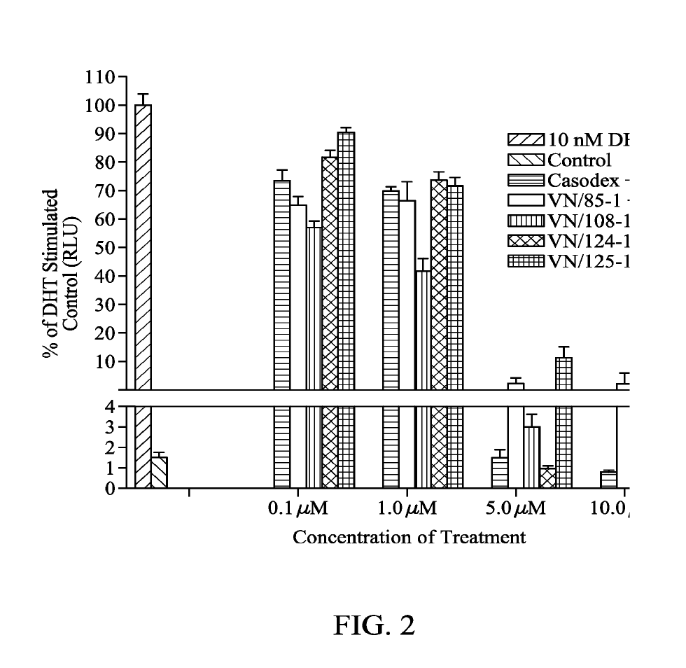

The compounds that showed strong binding affinity for the receptor were evaluated for antagonistic properties by the luciferase assay in LAPC4 and LNCaP cells transfected with the ARR2-Luc vector. These experiments were carried out against both receptor types, as there are reports of some wild-type AR antagonists, such as flutamide, functioning as T877A agonists [35, 36]. In both cell types, VN/124-1, VN/125-1 and VN/108-1 inhibited DHT induced transcriptional activation with similar potency as casodex. Casodex, VN/85-1, VN/124-1, and VN/125-1 at 10 μM concentration were all able to reduce WT and T877A AR-mediated transcriptional activation by 90-99% (FIG. 2). In LNCaP cells, VN/87-1 was the least effective of the compounds tested. When LNCaP-CYP17 cells were exposed to inhibitors in steroid free media, only VN/87-1 activated luciferase transcription, indicating that it is a partial agonist of the T877A AR, similar to flutamide. None of the other compounds displayed agonistic properties in either cell line.

AR Downregulation:

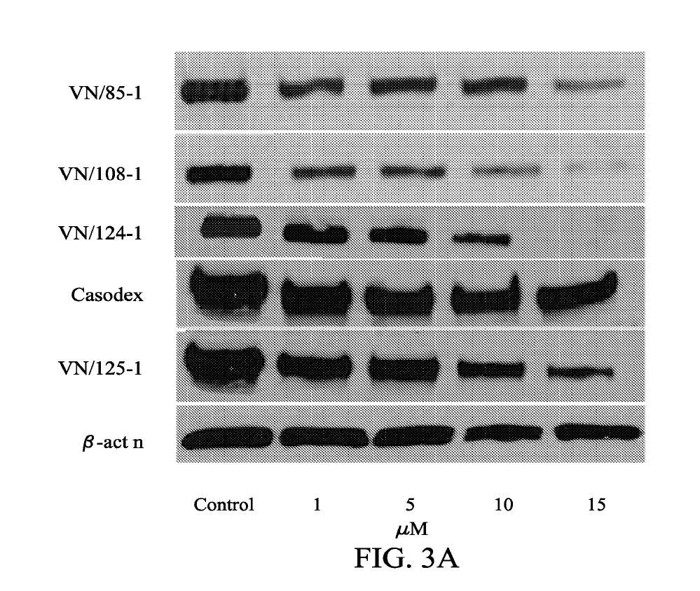

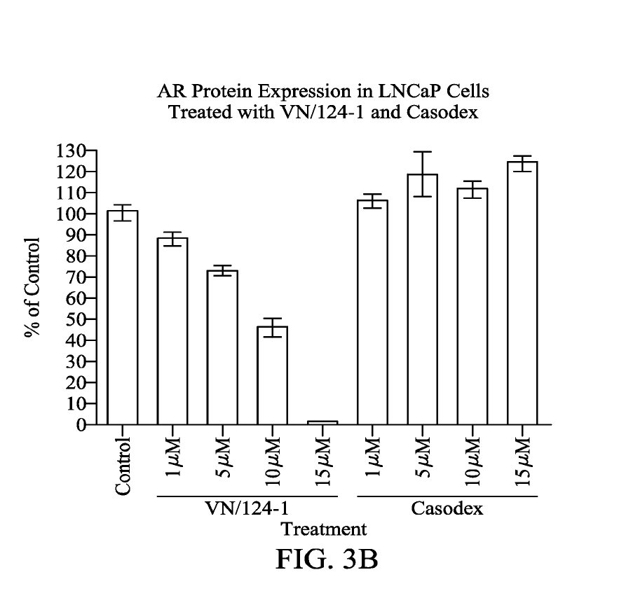

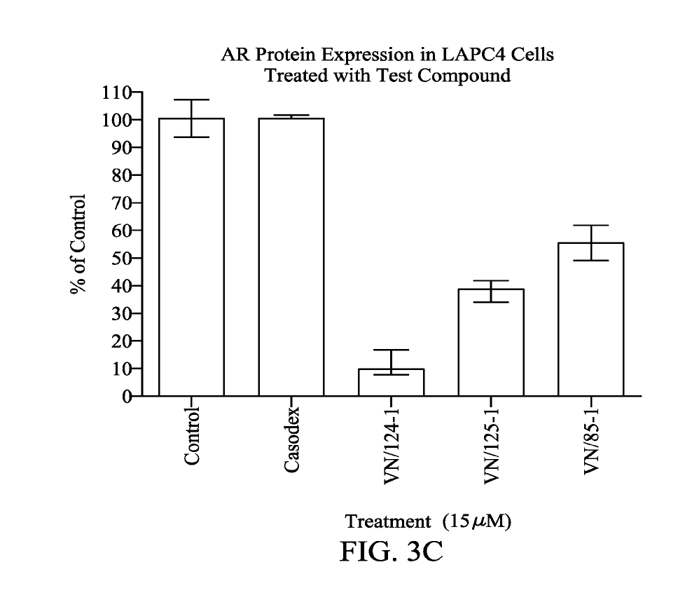

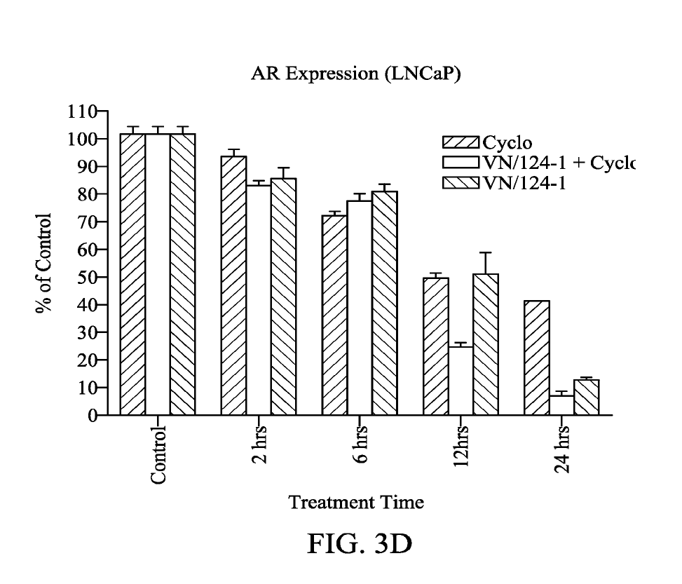

The compounds showed a dramatic down-regulation of wild-type and also mutated AR receptor. In LNCaP cells, nearly all of the test compounds induced a dose-dependent (1, 5, 10, 15 μM) decrease in AR levels whereas no change in total AR level was observed with casodex at these concentrations (FIGS. 3A-B). VN/124-1 reduced expression by 50% at 10 μM, and displayed nearly complete suppression at 15 μM. VN/125-1, VN/85-1 and VN/108-1 were able to reduce AR protein expression by 65%, 70% and 90% respectively at a concentration of 15 μM. In LAPC4 cells, VN/124-1 reduced expression by 89% at a 15 μM concentration. At the same concentration, VN/85-1 and VN/125-1 reduced expression by 50% and 66%, respectively (FIG. 3C).

AR Degradation:

Although VN/124-1 was able to down-regulate the AR protein expression in a dose-dependent manner, it was still unclear whether the down-regulation was a result of decreased protein synthesis or increased degradation/AR destabilization. To determine protein degradation, de novo protein synthesis was inhibited using cyclohexamide and protein expression was measured at various time points.

Cycloheximide treatment alone reduced AR levels in a time dependent fashion, with 50% reduction observed at 12 hours and over 60% at 24 hours post treatment. VN/124-1 treatment did not alter AR degradation rate for the first 6 hours, however a rapid decline in AR level occurred between 6 and 12 hours post treatment, resulting in 50% less receptor than expressed at 6 hours, and 75% less than control. The observed difference between 12 and 24 hours followed a similar pattern in both cycloheximide and VN/124-1 groups, with only an additional decline of approximately 10% for each (FIG. 3D). These results demonstrate that VN/124-1 increases the degradation rate of the AR.

Inhibition of Cell Proliferation:

The ability of the compounds to inhibit proliferation with and without DHT stimulation in LAPC4 and LNCaP cells was examined. In contrast to LNCaP cells, LAPC4 cells did not exhibit strong stimulation in response to DHT. This is in agreement with reports by other investigators [37]. As such, there was minimal difference between inhibition of DHT stimulated vs. non-stimulated LAPC4 cells for all test compounds, with IC₅₀'s ranging from 1-7 μM (Table 2 below). VN/85-1 and VN/108-1 were able to reduce cell proliferation in a consistent dose-dependent manner, with potency equal to or greater than casodex. VN/124-1, and VN/125-1 were also highly effective, with IC₅₀'s of 3.2 and 1.0 μM, as previously reported [28]. The time course to maximal effectiveness was similar among all test compounds, with onset of cell death being visually apparent no earlier than 48-72 hours post-treatment.

TABLE 2
Effect of Novel Compounds on Cell Proliferation

| | |
|---|---|
| LNCaP | LAPC4 |
| IC₅₀ (μM) | IC₅₀ (μM) |

DHT

| Compound | + | − | + | − |
|---|---|---|---|---|
| VN/85-1 | 3.7 | 1.9 | 4.2 | 3.4 |
| VN/87-1 | 4.8 | NT | NT | NT |
| VN/108-1 | 1.8 | 1.6 | 7 | 5 |
| VN/124-1 | 6 | 2.6 | 3.2 | 4 |
| VN/125-1 | 1.8 | 2.2 | 1.0 | 3 |
| Casodex | 8.6 | 4.6 | 10 | 9 |
| Flutamide | * | * | NT | NT |

Table 2 demonstrates the effect of compounds on PC cell proliferation. LNCaP cells were seeded at 15,000 cells/well in 24 well multi-well plates, and LAPC4 cells were seeded at 15,000 cells/well. Cells were treated with the indicated concentration of compound in steroid free medium with or without 1 nM DHT (LNCaP), or 10 nM DHT (LAPC4) and allowed to grow for 7 days. The number of viable cells was compared by MTT assay (LAPC4) or XTT assay (LNCaP) on the 7^{th} day. NT=not tested, NI=less than 50% inhibition at 20 μM, *=stimulated proliferation

Previous results with VN/85-1, VN/87-1, and VN/108-1 have shown significant inhibition of LNCaP cell proliferation. All three compounds inhibited proliferation by 40-60%, and inhibited DHT stimulated proliferation at concentrations up to 5 μM [33]. Further evaluation of these compounds in DHT stimulated LNCaP cells, over a broader concentration range of (0.01-100 μM), indicated IC₅₀ values of 1.8, 4.6 and 3.7 μM for VN/108-1, VN/85-1, and VN/87-1 respectively. To our knowledge, LNCaP cells do not express CYP17, or express very minimal amounts, as CYP17 activity is undetectable in LNCaP cells by our acetic acid releasing assay system (AARA). Therefore LNCaP viability assays do not completely represent the extent of our novel compounds' potential effectiveness, as under physiological conditions there would be the added effect of decreased androgen production. The fact that these compounds are equally effective against both cell lines indicates increased clinical potential, as some anti-androgens, such as flutamide, have agonistic properties for the mutant AR as occurs in LNCaP cells.

VN/124-1 Causes Growth Inhibition in LAPC4 Xenograft Model:

The effects of VN/124-1 were determined on prevention of LAPC4 tumor xenograft formation and also the effect of VN/124-1, VN/124-1+castration, castration or casodex on tumor growth in vivo. LAPC4 cells were injected s.c. into SCID mice and one group of mice (n=5; tumor prevention group) was treated with VN/124-1 (0.13 mmol/kg/twice daily) for 93 days starting the day after inoculation with LAPC4 cells. Approximately 9 weeks after inoculation tumors had formed in the other mice (approx 300 mm³), and these animals were assigned to five treatment groups: control (vehicle), castration, casodex (0.13 mmol/kg/twice daily), VN/124-1 (0.13 mmol/kg/twice daily) and castration plus VN/124-1 (0.13 mmol/kg/twice daily). It should be noted that experiments with the control and casodex groups were terminated on day 86 because of large tumors and drug shortage, respectively (FIG. 4). Treatment with VN/124-1 was very effective in preventing the formation of LAPC4 tumors (6.94 vs. 2410.28 mm³ in control group on day 86 (p<0.001)). Total tumor volume in the control mice increased by 4.3-fold over 3 weeks of treatment when the mice were sacrificed because of the large tumors.

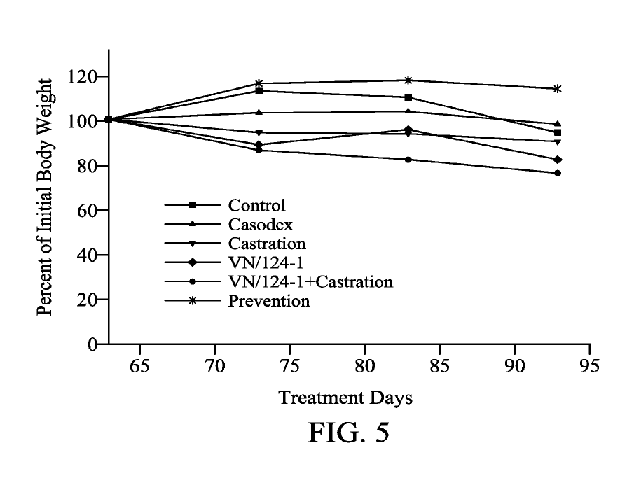

The tumors in the prevention group were significantly smaller than the tumors in control group and in all treatment groups (p<0.0001). Compared to control groups with the total tumor volume from day 76 to day 93 (effect after two weeks of treatment), all treatments were significantly effective after two weeks except for treatment with casodex (p=0.075). There were no significantly differences between treatment groups overall. Comparing the total tumor volumes at day 93, there were significant differences among all groups but there were no significant differences between castration and VN/124-1 groups (p=0.33) and between the VN/124-1 group and the VN/124-1+castration group (p=0.059). However, it was observed that in the castration and VN/124-1 groups, there was significant variation in tumor volumes due to reduced growth of tumors on either the left or right flanks of the mice. This resulted in large variations in the total tumor volumes for each group. However, when the two groups (i.e., castration vs. VN/124-1) were compared based on multivariate statistical analysis (allowing for differences in left tumor and right tumor) using F-test, there were significant differences between the tumor volumes of castration and VN/124-1 groups from day 76 to day 93 (p=0.031) and at day 93 (p=0.047). Furthermore, examination of the changes in average tumor volumes in all groups clearly shows the effects of the various treatments on tumor growth. No significant changes in animal body weights were observed in all treatment groups (FIG. 5), demonstrating that the treatments did not produce general toxicity in the mice.

Effects of Treatments on Androgen Receptor Expression In Vivo:

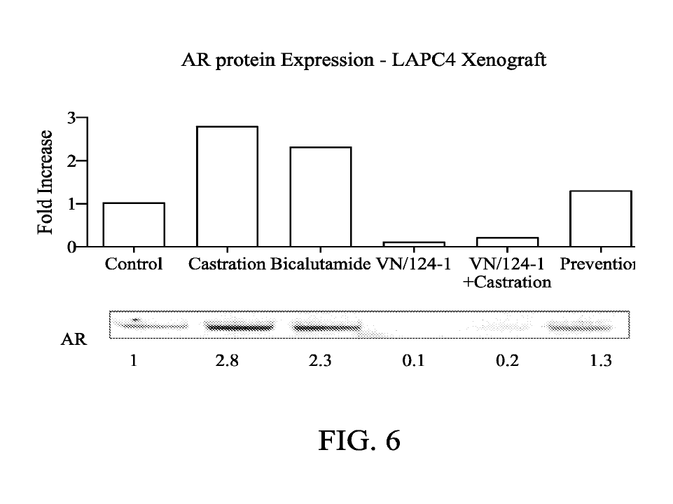

The striking difference in AR down-regulation between VN/124-1 and the other compounds, some of which had similar or better in vitro anti-proliferation, anti-androgen and lyase inhibition profiles, correlated with VN/124-1's increased activity in reducing LAPC4 tumor xenograft growth. Therefore, VN/124-1 treated LAPC4 tumor xenografts were analyzed for AR expression to determine if VN/124-1 maintained its potent down-regulation properties in vivo (FIG. 6). Analysis of tumors revealed that treatments with VN/124-1 or VN/124-1+castration caused marked reduction in AR protein of 10- and 5-fold, respectively. In contrast, treatment with bicalutamide or castration caused significant AR protein up-regulation of 2.3- and 2.8-fold, respectively. Treatment with VN/124-1 in the tumor-formation prevention study group caused a slight up-regulation (1.3-fold) of AR protein expression.

### CONCLUSION

The present inventors have previously reported that some CYP17 inhibitors act as anti-androgens against the LNCaP AR [15, 27, 38]. The affinity of the compounds to the AR was assessed in competitive binding studies carried out using the synthetic ligand methyltrienolone with the wild-type and two mutant forms of the AR. To determine if the compounds are agonists or antagonists, luciferase assays were performed in the presence and absence of DHT. The binding affinities of the compounds were strongest for the wild-type AR, with IC₅₀'s ranging from 248 nM (VN/85-1). Although the strongest affinity was 10-fold weaker than DHT (22 nM) in the same system, it is still significantly stronger than the clinically used antiandrogens casodex (4.3 μM). Abiraterone, a CYP17 inhibitor now in clinical trials [17] did not bind to the AR. In LNCaP cells (T877A mutation), VN/85-1, VN/124-1, and VN/125-1 had lower affinities, whereas VN/108-1, VN/87-1 and flutamide had affinities equivalent to those for the wild type AR. Casodex displayed a much stronger affinity for the T877A AR (971 nM), which was approximately the same as VN/124-1 (845 nM) and VN/108-1 (831 nM). Conversely, all of the compounds tested against the T575A mutant, which has a mutation in the DNA binding domain [34], displayed affinities equivalent to the wild-type AR. Mutations in the AR's ligand binding domain have been shown to alter the affinity of ligands and anti-androgens. Bohl et al. reported a two-fold higher affinity of casodex for W741L, a LBD AR mutant, as compared to the wild-type AR [39]. This is similar to our results with casodex and the T877A mutant. Interestingly, a few of our steroidal compounds exhibited a reduced affinity for the mutant T877A AR, in contrast to casodex's increased affinity.

Recent evidence indicates that in the majority of PC cases, even in chemotherapy resistant disease, the AR is still expressed and required for growth [40-43]. It has also been shown that the AR can be activated by co-factors and other mechanisms independent of androgen levels [44-46]. In addition, it has been demonstrated that over-expression of AR in a castration-resistant xenograft model is consistent with observations in human clinical specimens, and over-expression of AR promotes the transition from a hormone-dependent xenograft to a castration-resistant xenograft [5, 47]. These observations demonstrate an unfulfilled need in the art for directly targeting the AR and reducing AR levels to below a critical threshold so as to provide more effective approaches to treatment than current antiandrogens.

In contrast to casodex, the antiandrogen currently used clinically, the novel compounds VN/85-1, VN/108-1, VN/125-1 and VN/124-1 were all able to greatly reduce AR levels. This effect was observed in both LAPC4 cells and LNCaP cells, with overall AR levels decreased by 60% or more. In both cell lines, VN/124-1 was significantly more potent than the other compounds, with nearly complete reduction of AR expression at 15 μM in LNCaP cells, and 89% in LAPC4 cells. Analysis of LAPC4 tumor samples from xenografts revealed that VN/124-1 also reduced AR in vivo, with a marked decrease in AR as compared to castration and control tumors. Results of analyzing tumor samples from our former study of VN/124-1 in xenografts [28] that were not previously reported showed similar reduction in AR, confirming this mechanism of action of VN/124-1 in vivo (Data not shown). Although VN/85-1 and VN/125-1 had similar or better characteristics than VN/124-1 in terms of inhibiting the CYP17 and reducing androgen modulated transcription, they were much less effective in vivo. However, these results could be explained in part by the greater effect of VN/124-1 on reducing AR levels in vitro and in vivo. Additional in vitro evidence supports this view, as reduction of AR expression produced a more pronounced effect on AR-induced transcription and cell growth than androgen deprivation in two androgen-insensitive PC cell lines, LNCaP-C42B4 and CWR22Rv1 [10].

Without being bound by theory, the mechanism of AR down-regulation could occur through increased degradation or reduced protein synthesis. For VN/124-1, AR degradation patterns were examined to determine whether AR stability was being affected. Destabilization of the AR has been shown in steroid depleted conditions, with half-life reduced from approximately six hours to three hours [48]. By using cycloheximide to inhibit new protein synthesis, and measuring the rate of degradation, it was possible to determine if VN/124-1 caused additional degradation beyond that normally observed under androgen deprivation. There was reduction of 50% in AR levels in the VN/124-1 treatment group versus control cells 6 hrs post-treatment. AR levels continued to decline over 24 hours, with an additional 10% reduction over control evident at 12 and 24 hours post treatment. This data indicates that VN/124-1's down-regulation of the AR level is at least partly due to increased AR degradation. However, it should be noted that androgens have been shown to increase AR synthesis as well [48]. Therefore the possibility of an additional effect on modulating the rate of AR expression cannot be ruled out. Consequently, the effects of VN/124-1 on AR mRNA expression are being conducted. Also, the mechanism by which degradation occurs is still unknown. AR degradation has been shown to proceed through two proteolytic pathways. One relies on proteosomal degradation and occurs both in the absence (ligand-independent) or presence (ligand-dependent) of the hormone [reviewed by [49]]. The second engages PTEN and caspase-3 activity [50]. Interestingly, VN/124-1 is able to reduce AR levels in the presence and absence of androgens. Therefore, as long as the AR is functional, VN/124-1 may inhibit PC cell growth via AR down-regulation regardless of androgen-dependent or castration-resistant status.

In the LAPC4 xenograft, VN/124-1 is a more potent agent in reducing tumor growth than other compounds (VN/85, VN/87, and VN108) and is more effective than castration and casodex. VN/124-1 plus castration was also significantly better than castration alone or casodex. VN/124-1 was most effective at preventing the formation of LAPC4 tumor xenografts demonstrating its potential as a chemopreventive agent. It has been shown that unlike treatment with casodex or castration, which caused significant AR protein up-regulation, treatment with VN/124-1 markedly reduced AR protein levels both in vivo and in vitro. Without being bound by theory, this additional property may account for the superiority of VN/124-1 in vivo compared to other more potent CYP17 inhibitors such as VN/85-1.

Although the invention has been described in example embodiments, those skilled in the art will appreciate that various modifications may be made without departing from the spirit and scope of the invention. It is therefore to be understood that the inventions herein may be practiced other than as specifically described. Thus, the present embodiments should be considered in all respects as illustrative and not restrictive. Accordingly, it is intended that such modifications fall within the scope of the present invention as defined by the claims appended hereto.

### REFERENCES

1. Jemal, A., Siegel, R., Ward, E., Murray, T., Xu, J., Smigal, C., et al., Cancer statistics, 2006. CA Cancer J Clin, 2006. 56(2): p. 106-30.
2. McConnell, J. D., Physiological basis of endocrine therapy for prostatic cancer. Urol. Clin. North Am, 1991. 18: p. 1-13.
3. Bruchovsky, N. and Wilson, J. D., The conversion of testosterone to 5-alpha-androstan-17-beta-ol-3-one by rat prostate in vivo and in vitro. J Biol Chem, 1968. 243(8): p. 2012-21.
4. Crawford, E. D., Eisenberger, M. A., McLeod, D. G., Spaulding, J. T., Benson, R., Dorr, F. A., Blumstein, B. A., Davis, M. A., and Goodman, P. J., A controlled trial of leuprolide with and without flutamide in prostatic carcinoma. N. Eng. J. Med., 1989. 321: p. 419-424.
5. Chen, C. D., Welsbie, D. S., Tran, C., Baek, S. H., Chen, R., Vessella, R., et al., Molecular determinants of resistance to antiandrogen therapy. Nat Med, 2004. 10(1): p. 33-9.
6. Taplin, M. E., Bubley, G. J., Shuster, T. D., Frantz, M. E., Spooner, A. E., Ogata, G. K., et al., Mutation of the androgen-receptor gene in metastatic androgen-independent prostate cancer. N Engl J Med, 1995. 332(21): p. 1393-8.
7. Tilley, W. D., Bentel, J. M., Aspinall, J. O., Hall, R. E., and Horsfall, D. J., Evidence for a novel mechanism of androgen resistance in the human prostate cancer cell line, PC-3. Steroids, 1995. 60(1): p. 180-6.
8. Tilley, W. D., Buchanan, G., Hickey, T. E., and Bentel, J. M., Mutations in the androgen receptor gene are associated with progression of human prostate cancer to androgen independence. Clin Cancer Res, 1996. 2(2): p. 277-85.
9. Visakorpi, T., Hyytinen, E., Koivisto, P., Tanner, M., Keinanen, R., Palmberg, C., et al., In vivo amplification of the androgen receptor gene and progression of human prostate cancer. Nat Genet, 1995. 9(4): p. 401-6.
10. Li, T. H., Zhao, H., Peng, Y., Beliakoff, J., Brooks, J. D., and Sun, Z., A promoting role of androgen receptor in androgen-sensitive and -insensitive prostate cancer cells. Nucleic Acids Res, 2007. 35(8): p. 2767-76.
11. Yuan, X., Li, T., Wang, H., Zhang, T., Barua, M., Borgesi, R. A., et al., Androgen receptor remains critical for cell-cycle progression in androgen-independent CWR22 prostate cancer cells. Am J Pathol, 2006. 169(2): p. 682-96.
12. Cha, T. L., Qiu, L., Chen, C. T., Wen, Y., and Hung, M. C., Emodin down-regulates androgen receptor and inhibits prostate cancer cell growth. Cancer Res, 2005. 65(6): p. 2287-95.
13. Bhuiyan, M. M., Li, Y., Banerjee, S., Ahmed, F., Wang, Z., Ali, S., et al., Down-regulation of androgen receptor by 3,3′-diindolylmethane contributes to inhibition of cell proliferation and induction of apoptosis in both hormone-sensitive LNCaP and insensitive C4-2B prostate cancer cells. Cancer Res, 2006. 66(20): p. 10064-72.
14. Mostaghel, E. A., Page, S. T., Lin, D. W., Fazli, L., Coleman, I. M., True, L. D., et al., Intraprostatic androgens and androgen-regulated gene expression persist after testosterone suppression: therapeutic implications for castration-resistant prostate cancer. Cancer Res, 2007. 67(10): p. 5033-41.
15. Suzuki, K., Nishiyama, T., Hara, N., Yamana, K., Takahashi, K., and Labrie, F., Importance of the intracrine metabolism of adrenal androgens in androgen-dependent prostate cancer. Prostate Cancer Prostatic Dis, 2007. 10(3): p. 301-6.
16. Stanbrough, M., Bubley, G. J., Ross, K., Golub, T. R., Rubin, M. A., Penning, T. M., et al., Increased expression of genes converting adrenal androgens to testosterone in androgen-independent prostate cancer. Cancer Res, 2006. 66(5): p. 2815-25.
17. Hall, P. F., Cytochrome P-450 C21scc: one enzyme with two actions: hydroxylase and lyase. J Steroid Biochem Mol Biol, 1991. 40(4-6): p. 527-32.
18. Trachtenberg, J., Halpern, N., and Pont, A., Ketoconazole: a novel and rapid treatment for advanced prostatic cancer. J Urol, 1983. 130(1): p. 152-3.
19. Small, E. J., Baron, A. D., Fippin, L., and Apodaca, D., Ketoconazole retains activity in advanced prostate cancer patients with progression despite flutamide withdrawal. J Urol, 1997. 157(4): p. 1204-7.
20. Njar, V. C. and Brodie, A. M., Inhibitors of 17alpha-hydroxylase/17,20-lyase (CYP17): potential agents for the treatment of prostate cancer. Curr Pharm Des, 1999. 5(3): p. 163-80.
21. Hartmann, R. W., Ehmer, P. B., Haidar, S., Hector, M., Jose, J., Klein, C. D., et al., Inhibition of CYP 17, a new strategy for the treatment of prostate cancer. Arch Pharm (Weinheim), 2002. 335(4): p. 119-28.
22. Hakki, T. and Bernhardt, R., CYP17- and CYP11B-dependent steroid hydroxylases as drug development targets. Pharmacol Ther, 2006. 111(1): p. 27-52.
23. Leroux, F., Inhibition of p450 17 as a new strategy for the treatment of prostate cancer. Curr Med Chem, 2005. 12(14): p. 1623-9.
24. O'Donnell, A., Judson, I., Dowsett, M., Raynaud, F., Dearnaley, D., Mason, M., et al., Hormonal impact of the 17alpha-hydroxylase/C(17,20)-lyase inhibitor abiraterone acetate (CB7630) in patients with prostate cancer. Br J Cancer, 2004. 90(12): p. 2317-25.
25. Attard, G., Belldegrun, A. S., and de Bono, J. S., Selective blockade of androgenic steroid synthesis by novel lyase inhibitors as a therapeutic strategy for treating metastatic prostate cancer. BJU Int, 2005. 96(9): p. 1241-6.
26. Njar, V. C., Kato, K., Nnane, I. P., Grigoryev, D. N., Long, B. J., and Brodie, A. M., Novel 17-azolyl steroids, potent inhibitors of human cytochrome 17 alpha-hydroxylase-C17,20-lyase (P450(17) alpha): potential agents for the treatment of prostate cancer. J Med Chem, 1998. 41(6): p. 902-12.
27. Long, B. J., Grigoryev, D. N., Nnane, I. P., Liu, Y., Ling, Y. Z., and Brodie, A. M., Antiandrogenic effects of novel androgen synthesis inhibitors on hormone-dependent prostate cancer. Cancer Res, 2000. 60(23): p. 6630-40.
28. Handratta, V. D., Vasaitis, T. S., Njar, V. C., Gediya, L. K., Kataria, R., Chopra, P., et al., Novel C-17-heteroaryl steroidal CYP17 inhibitors/antiandrogens: synthesis, in vitro biological activity, pharmacokinetics, and antitumor activity in the LAPC4 human prostate cancer xenograft model. J Med Chem, 2005. 48(8): p. 2972-84.
29. Handratta, V. D., Jelovac, D., Long, B. J., Kataria, R., Nnane, I. P., Njar, V. C., et al., Potent CYP17 inhibitors: improved syntheses, pharmacokinetics and anti-tumor activity in the LNCaP human prostate cancer model. J Steroid Biochem Mol Biol, 2004. 92(3): p. 155-65.
30. Guo, Z., Dai, B., Jiang, T., Xu, K., Xie, Y., Kim, O., et al., Regulation of androgen receptor activity by tyrosine phosphorylation. Cancer Cell, 2006. 10(4): p. 309-19.
31. Marcelli, M., Ittmann, M., Mariani, S., Sutherland, R., Nigam, R., Murthy, L., et al., Androgen receptor mutations in prostate cancer. Cancer Res, 2000. 60(4): p. 944-9.
32. Tan, M., Fang, H. B., Tian, G. L., and Houghton, P. J., Small-sample inference for incomplete longitudinal data with truncation and censoring in tumor xenograft models. Biometrics, 2002. 58(3): p. 612-20.
33. Grigoryev, D. N., Long, B. J., Nnane, I. P., Njar, V. C., Liu, Y., and Brodie, A. M., Effects of new 17alpha-hydroxylase/C(17,20)-lyase inhibitors on LNCaP prostate cancer cell growth in vitro and in vivo. Br J Cancer, 1999. 81(4): p. 622-30.
34. Veldscholte, J., Ris-Stalpers, C., Kuiper, G. G., Jenster, G., Berrevoets, C., Claassen, E., et al., A mutation in the ligand binding domain of the androgen receptor of human LNCaP cells affects steroid binding characteristics and response to anti-androgens. Biochem Biophys Res Commun, 1990. 173(2): p. 534-40.
35. Culig, Z., Hoffmann, J., Erdel, M., Eder, I. E., Hobisch, A., Hittmair, A., et al., Switch from antagonist to agonist of the androgen receptor bicalutamide is associated with prostate tumour progression in a new model system. Br J Cancer, 1999. 81(2): p. 242-51.
36. Steketee, K., Timmerman, L., Ziel-van der Made, A. C., Doesburg, P., Brinkmann, A. O., and Trapman, J., Broadened ligand responsiveness of androgen receptor mutants obtained by random amino acid substitution of H874 and mutation hot spot T877 in prostate cancer. Int J Cancer, 2002. 100(3): p. 309-17.
37. Thompson, T. A. and Wilding, G., Androgen antagonist activity by the antioxidant moiety of vitamin E, 2,2,5,7,8-pentamethyl-6-chromanol in human prostate carcinoma cells. Mol Cancer Ther, 2003. 2(8): p. 797-803.
38. Klus, G. T., Nakamura, J., Li, J. S., Ling, Y. Z., Son, C., Kemppainen, J. A., et al., Growth inhibition of human prostate cells in vitro by novel inhibitors of androgen synthesis. Cancer Res, 1996. 56(21): p. 4956-64.
39. Bohl, C. E., Gao, W., Miller, D. D., Bell, C. E., and Dalton, J. T., Structural basis for antagonism and resistance of bicalutamide in prostate cancer. Proc Natl Acad Sci USA, 2005. 102(17): p. 6201-6.
40. Feldman, B. J. and Feldman, D., The development of androgen-independent prostate cancer. Nat Rev Cancer, 2001. 1(1): p. 34-45.
41. Grossmann, M. E., Huang, H., and Tindall, D. J., Androgen receptor signaling in androgen-refractory prostate cancer. J Natl Cancer Inst, 2001. 93(22): p. 1687-97.
42. Arnold, J. T. and Isaacs, J. T., Mechanisms involved in the progression of androgen-independent prostate cancers: it is not only the cancer cell's fault. Endocr Relat Cancer, 2002. 9(1): p. 61-73.
43. Zegarra-Moro, O. L., Schmidt, L. J., Huang, H., and Tindall, D. J., Disruption of androgen receptor function inhibits proliferation of androgen-refractory prostate cancer cells. Cancer Res, 2002. 62(4): p. 1008-13.
44. Yeh, S., Kang, H. Y., Miyamoto, H., Nishimura, K., Chang, H. C., Ting, H. J., et al., Differential induction of androgen receptor transactivation by different androgen receptor coactivators in human prostate cancer DU145 cells. Endocrine, 1999. 11(2): p. 195-202.
45. Yeh, S., Miyamoto, H., Shima, H., and Chang, C., From estrogen to androgen receptor: a new pathway for sex hormones in prostate. Proc Natl Acad Sci USA, 1998. 95(10): p. 5527-32.
46. Gregory, C. W., He, B., Johnson, R. T., Ford, O. H., Mohler, J. L., French, F. S., et al., A mechanism for androgen receptor-mediated prostate cancer recurrence after androgen deprivation therapy. Cancer Res, 2001. 61(11): p. 4315-9.
47. Linja, M. J., Savinainen, K. J., Saramaki, O. R., Tammela, T. L., Vessella, R. L., and Visakorpi, T., Amplification and overexpression of androgen receptor gene in hormone-refractory prostate cancer. Cancer Res, 2001. 61(9): p. 3550-5.
48. Syms, A. J., Norris, J. S., Panko, W. B., and Smith, R. G., Mechanism of androgen-receptor augmentation. Analysis of receptor synthesis and degradation by the density-shift technique. J Biol Chem, 1985. 260(1): p. 455-61.
49. Jaworski, T., Degradation and beyond: Control of androgen receptor activity by the proteasome system. Cell Mol Biol Lett, 2006. 11(1): p. 109-31.
50. Lin, H. K., Hu, Y. C., Lee, D. K., and Chang, C., Regulation of androgen receptor signaling by PTEN (phosphatase and tensin homolog deleted on chromosome 10) tumor suppressor through distinct mechanisms in prostate cancer cells. Mol Endocrinol, 2004. 18(10): p. 2409-23.

1-20. (canceled)

30. A method comprising steps of:
contacting one or more prostate tumor cells whose androgen receptor level is known with a compound of Formula I, 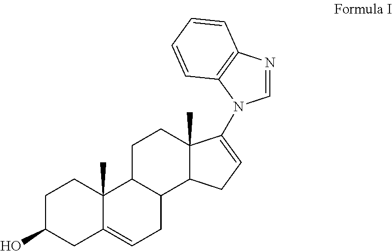 [SMILES: C[C@]12CC[C@H](O)CC1=CCC1C2CC[C@]2(C)C(n3cnc4ccccc43)=CCC12]
or a pharmaceutically acceptable salt thereof,
in an amount and for a time sufficient such that the level is reduced by at least about 50%.

31. The method of claim 30, wherein the prostate tumor cells comprise castration-resistant prostate tumor cells.

32. The method of claim 30, wherein the androgen receptors comprise wild-type androgen receptors.

33. The method of claim 30, wherein the wild-type androgen receptors comprise mutant androgen receptors.

34. The method of claim 33, wherein the mutant androgen receptors comprise a mutation in a ligand binding domain of the receptor.

35. The method of claim 34, wherein a mutation in the ligand binding domain of the receptor is a T877A mutation.

36. The method of claim 30, wherein proliferation of the prostate tumor cells is reduced.

37. The method of claim 30, wherein growth of the prostate tumor cells is reduced.

38. A method comprising steps of:
administering to a subject, having one or more prostate tumor cells whose androgen receptor level is known, a compound of Formula I, 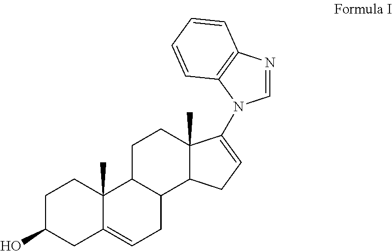 [SMILES: C[C@]12CC[C@H](O)CC1=CCC1C2CC[C@]2(C)C(n3cnc4ccccc43)=CCC12]
or a pharmaceutically acceptable salt thereof,
in an amount and for a time sufficient such that the level is reduced by at least about 50%.

39. The method of claim 38, wherein the prostate tumor cells comprise castration-resistant prostate tumor cells.

40. The method of claim 38, wherein the androgen receptors comprise wild-type androgen receptors.

41. The method of claim 38, wherein the wild-type androgen receptors comprise mutant androgen receptors.

42. The method of claim 41, wherein the mutant androgen receptors comprise a mutation in a ligand binding domain of the receptor.

43. The method of claim 38, wherein a mutation in the ligand binding domain of the receptor is a T877A mutation.

44. The method of claim 38, wherein proliferation of the prostate tumor cells is reduced.

45. The method of claim 38, wherein growth of the prostate tumor cells is reduced.

46. The method of claim 38, wherein the subject is human.
